(19) United States
(12) Patent Application Publication
(10) Pub. No.: US 2018/0335427 A1
(43) Pub. Date: Nov. 22, 2018

(54) Tags for Identifying and Tracking Pharmaceutical and Nutritional Products using Paramagnetic Microparticles and Detectable Chemicals

(71) Applicants: David R. Hall, Provo, UT (US); Steven J.M. Butala, Provo, UT (US); Dan Allen, Springville, UT (US); Andrew Nguyen, Provo, UT (US); Conrad Rosenbrock, Provo, UT (US); Ben Swenson, Lehi, UT (US); Daniel Hendricks, Provo, UT (US); Travis Niederhauser, Mapleton, UT (US); Terrece Pearman, Draper, UT (US); Joe Fox, Spanish Fork, UT (US)

(72) Inventors: David R. Hall, Provo, UT (US); Steven J.M. Butala, Provo, UT (US); Dan Allen, Springville, UT (US); Andrew Nguyen, Provo, UT (US); Conrad Rosenbrock, Provo, UT (US); Ben Swenson, Lehi, UT (US); Daniel Hendricks, Provo, UT (US); Travis Niederhauser, Mapleton, UT (US); Terrece Pearman, Draper, UT (US); Joe Fox, Spanish Fork, UT (US)

(21) Appl. No.: 15/601,532

(22) Filed: May 22, 2017

(51) Int. Cl. G01N 33/543; C12Q 1/68; G01N 33/94

(52) U.S. Cl. CPC G01N 33/54326; G01N 33/94; C12Q 1/6834

(57) ABSTRACT

We disclose a tag for tracking and identifying pharmaceutical and nutritional products. The tag includes a paramagnetic microparticle which is connected to at least one unique and detectable chemical. The chemical may be coated on the paramagnetic microparticle or connected through functional groups. The tags may be too large to be taken into the bloodstream and therefore remain in the user's gastrointestinal tract. The tags may be fully or partially isolated from feces for analysis. The tags are attracted to an external electromagnetic force but are not magnetically attracted to each other. Consequently, the tags are safe to ingest. The tags may vary in volume or mass so as to be separable according to mass. The tags may be non-spherical in shape thus increasing the surface area to volume ratio and increasing the amount of chemical which may be attached as a taggant.

### BACKGROUND

### Field of the Invention

This disclosure relates to tags for pharmaceutical and nutritional products for use in identifying and distinguishing the products.

### Background of the Invention

Detecting and verifying consumption of pharmaceutical or nutritional products is a challenge for managing product distribution, patient treatment, drug compliance in patients and clinical trial subjects, drug abuse, counterfeit products, and confirming food ingredients and sources. Some active ingredients in pharmaceutical or nutritional products are delivered in quantities as small as 10-20 mg or even smaller. These small quantities make direct detection of the active ingredient challenging. Furthermore, many pharmaceutical or nutritional products are metabolized or partially metabolized in the body. Consequently, the concentration of the active ingredient and the concentration of metabolic by-products of the active ingredient in biological samples varies over time.

Several methods have been used to identify consumed pharmaceutical or nutritional products in bodily waste. These methods include micro-scale consumable bar codes which pass through the digestive tract, unique surface markings, and unique combinations of pill shapes and colors. Detection of these unique features urine or feces is not possible in some cases.

Chemical taggants have been used to a limited degree. These taggants are limited because components of pills must be sufficiently biologically inert so that they do not adversely affect the delivery or activity of the active ingredients. A taggant optimized for properties such as optical or chemical detectability may not meet these requirements. Sample preparation and difficulty in detecting dilute levels of chemicals in biological samples may also present difficulties.

A series of tags that may be added to pharmaceutical or nutritional products and which are biologically inert, easily isolated and detected, and which are detectable in a biological sample is needed. Ideally, the tags may identify many data points along the chain of distribution.

### BRIEF SUMMARY OF THE INVENTION

We disclose a method of tagging pharmaceutical or nutritional products using paramagnetic microparticles which are connected to at least one unique and detectable chemical. The tags may include information about the pharmaceutical or nutritional products including a manufacturing site, a distributor, a dispensing pharmacy, a retailer, a prescribing healthcare provider, a formulation, a drug, a nutritional supplement, a food ingredient, and a prescribed end-user. The information may include the identity of the pharmaceutical or nutritional product.

The paramagnetic microparticles may be of a large enough diameter to be contained in the gastrointestinal tract upon ingestion with a tagged product. The paramagnetic microparticles do not have a magnetic force until exposed to an electromagnetic field. Consequently, unlike magnets, they are safe to ingest and may be isolated from feces after excretion by exposing the paramagnetic microparticles to a magnetic field gradient.

The unique and detectable chemicals may be coated onto the paramagnetic microparticles or associated with the paramagnetic microparticles through a functional group. Each paramagnetic microparticle may be connected to a single unique and detectable chemical or multiple unique and detectable chemicals.

The diameter of the paramagnetic microparticle may also act as a tag as well as assist in isolating different tags from each other. The magnetic force the paramagnetic microparticles experience in the presence of an electromagnetic field may be proportional to their diameter. Different diameters may be associated with different categories or other information about the product. The different diameters may also be used when isolating the tags from feces according to the diameters of their paramagnetic microparticles.

The paramagnetic microparticles within the disclosed tags may be a variety of non-spherical shapes. The non-spherical shapes may have an aspect ratio of between about 1:1 and 1:25. The non-spherical shapes may be those which increase the surface area to volume ratio of the paramagnetic microparticles relative to that of a spherical shape with the same volume. The increased surface area provides space to connect an increased number of chemicals to the paramagnetic microparticle. Consequently, chemicals with a greater minimum detection limit may be used to create the tags.

The unique and detectable chemicals connected to the paramagnetic microparticles may be analyzed using a variety of techniques known in the art, including, but not limited to, light absorption spectroscopy, Raman spectroscopy, surface plasmon resonance, fluorescence, electrochemical detection, ion chromatography and enzyme color change chemistry. The light absorption spectroscopy may include one or more of near infrared spectroscopy, visible spectroscopy, and ultraviolet spectroscopy. The analytical device, which may be a light absorption spectrometer, may be part of a medical toilet. The user may defecate into the medical toilet and tagged paramagnetic microparticles may be fully or partially isolated and analyzed within the medical toilet.

A protective coating such as a dielectric may be applied to the microparticle to protect both the particle and the chemical ligands. Alternatively, branched ligands or dendritic ligands may be attached to the microparticle to provide steric hindrance of chemical attack of surface-bound chemicals or chemical attack of the paramagnetic particle. The ligands themselves may be the detected chemical. In another example, the paramagnetic particle is coated by an inert shell, which is then coated by a chemical label or ligand. The inert shell may also comprise the detectable chemical. Examples of inert shells include oxides of silicon and nitrogen. Another example of a protective chemical is cellulose, which is not digestible by humans or other non-ruminant mammals.

### BRIEF DESCRIPTION OF THE DRAWINGS

FIG. 1 illustrates a cross-sectional view of a series of tags including paramagnetic microparticles of different sizes.

FIG. 2 illustrates the order in which the tags of FIG. 1 may be separated when exposed to an external electromagnetic force.

FIG. 3A illustrates a cross-sectional view of a paramagnetic microparticle which is connected to a polynucleotide encoded with information about a pharmaceutical or nutritional product.

FIG. 3B illustrates an expanded view of the polynucleotide connected to the paramagnetic microparticle of FIG. 3A.

FIG. 4 illustrates a cross-sectional view of an embodiment of a paramagnetic microparticle which is connected to multiple chemicals, each associated with a unique piece of information.

FIG. 5 illustrates a cross-sectional view of an embodiment of a tag which includes a paramagnetic microparticle coated with a ligand.

FIG. 6 illustrates cross-sectional view of an embodiment of a tag which includes a paramagnetic microparticle coated with a binding protein which binds the ligand of FIG. 5.

FIG. 7 illustrates relative surface area to volume ratios of three dimensional shapes which the paramagnetic microparticles may comprise.

FIG. 8 illustrates embodiments of shapes which the paramagnetic microparticles of the disclosed tags may comprise.

FIG. 9A illustrates a cross-sectional view of a tag including a paramagnetic microparticle with villi-shaped extensions and including single-stranded DNA as in FIG. 3B.

FIG. 9B illustrates a cross-sectional view of a functionalized tag including a paramagnetic microparticle with villi-shaped extensions and with chemicals connected to the functional groups.

FIG. 9C illustrates a perspective view of the tag of FIG. 9A.

FIG. 10 illustrates a tag that includes a porous paramagnetic microparticle with an amorphous shape according to an embodiment of the disclosure.

### DETAILED DESCRIPTION OF THE INVENTION

### Definitions

Drug, as used herein, means any pharmacologically or physiologically active agent or mixture of agents. Drug may also include an active ingredient in a health product, including a nutritional supplement. Drug may include one or more placebos.

User, as used herein, means a patient, a participant in a medical study, or any individual who has consumed a pharmaceutical or nutritional product which includes at least one taggant as described herein. The user may be animal or human.

Medical toilet, as used herein, means a device that may be used to collect one or more biological sample from a user. This may include a traditional water toilet. However, medical toilet, as used herein, may mean any device which may be used to collect biological samples according to the present disclosure and which may be equipped to analyze feces and other biological samples.

Nutritional product, as used herein, means a nutritional supplement, food, food ingredient, or any product from which a user may derive nutrients.

Shape factor, as used herein, means the ratio of the surface area of a non-spherical microparticle to that of a spherical microparticle, where both of the microparticles have identical volumes. Shape factor is explained in detail by Qi, W. H., Wang, M. P., and Liu, Q. H., (2005) Shape factor of nonspherical nanoparticles. J. Mater. Sci. 40, 2737-2739 which is hereby incorporated by reference in its entirety. Additionally, the non-spherical shapes may have an aspect ratio of between about 1:1 and 1:25.

While this invention is susceptible of embodiment in many different forms, there are shown in the drawings, which will herein be described in detail, several specific embodiments with the understanding that the present disclosure is to be considered as an exemplification of the principals of the invention and is not intended to limit the invention to the illustrated embodiments.

We disclose a tag which may be used to identify pharmaceutical or nutritional products. The tag includes at least one paramagnetic microparticle which may be connected to one or more chemicals. Each chemical may be associated with information about the products, including categories into which the products have been divided and specific information about the products, including the product identity. Each of the unique and detectable chemicals may be associated with a different category or specific information about each product.

The categories and information may include, but are not limited to, a manufacturing site, a distributor, a dispensing pharmacy, a retailer, a prescribing healthcare provider, a formulation, a drug, a nutritional supplement, a food ingredient, and a prescribed end-user. The information may include the identity of the pharmaceutical or nutritional product.

The paramagnetic microparticles and their unique and detectable chemicals may be metabolically stable. Often, the tagged products are intended to be taken internally. For example, the products may be swallowed and enter the gastrointestinal tract. Consequently, the paramagnetic microparticles and their unique and detectable taggants may be stable after complete passage through a user's gastrointestinal tract.

The paramagnetic microparticles are attracted to an electromagnetic force, but not to each other. Consequently, unlike magnets, the paramagnetic microparticles are safe to ingest. Only upon excretion are the paramagnetic microparticles intended to be exposed to an electromagnetic force. The electromagnetic force may then be used to completely or partially isolate the tags from the user's feces. The unique and detectable chemicals which are connected to the paramagnetic microparticles may then be analyzed and identified to obtain the information or category associated with them.

The volume of the paramagnetic microparticles may be large enough that they are not taken into the bloodstream and, consequently, are not excreted in a user's urine. The disclosed paramagnetic microparticles may be completely excreted in the user's feces. The paramagnetic microparticles may be completely or partially isolated from the user's feces and analyzed. In some embodiments, the paramagnetic microparticles may have a diameter at their widest point from between about 3 μm and about 300 μm. In some embodiments, their diameter at their widest point may be between about 5 μm and about 300 μm and in others, between about 10 μm and about 100 μm. In some embodiments, their diameter at their widest point may be between about 5 μm and about 15 μm. In some embodiments, the diameter of the paramagnetic microparticle at its widest point may be about 1 mm.

In some embodiments, the tags may include a plurality of paramagnetic microparticles of different sizes. The size may vary with regard to mass, volume, and diameter at the widest point of the paramagnetic microparticle. The different sizes of the paramagnetic microparticles may be used to distinguish different tags. More specifically, each mass, diameter, or volume of the paramagnetic microparticle may be associated with a different category as discussed herein. Consequently, both the chemical connected to the paramagnetic microparticle and the paramagnetic microparticle itself impart information about the tagged product.

Each paramagnetic particle of a certain mass, diameter, or volume may be connected to a different unique and detectable chemical. Alternatively, each paramagnetic particle of a certain mass, diameter, or volume may be connected to a plurality of different unique and detectable chemicals. In some embodiments, the plurality of different chemicals may be a set of chemicals, each associated with a set of related categories or pieces of information.

The paramagnetic microparticles with different sizes may have different magnetic forces in the presence of an external electromagnetic force. The larger paramagnetic microparticles may experience a stronger magnetic force in the presence of an electromagnetic force than smaller paramagnetic microparticles. Consequently, the paramagnetic microparticles may be fully or partially isolated from each other as a function of their respective sizes. The paramagnetic microparticles may then be divided into different pools based on their respective sizes. Both the sizes of the paramagnetic particles and their associated unique and detectable chemicals may be associated with categories or other information about the product.

Tags which include larger paramagnetic microparticles may include non-immunogenic coatings which may prevent an immune response to the tag when a user consumes the tag. In some embodiments, the non-immunogenic coating may include polyethylene glycol (PEG), poly(lactic-coglycolic acid) (PLGA), dendrimers, chitosan, sodium alginate, polyvinylpyrrolidone (PVP), copovidone, methylcellulose, cyclomethicones, or gold.

The non-spherical shapes may have an aspect ratio of between about 1:1 and 1:25. Additionally, the paramagnetic microparticles may comprises of a variety of non-spherical shapes which may increase the surface area to volume ratio relative to a spherical particle of the same volume. A greater surface area to volume ratio provides more surface for connecting chemicals to the tag for a particle of a certain volume. By increasing the number of molecules of a chemical which may be connected to a paramagnetic microparticle, a chemical with a lower detection limit may be used in the tag. Consequently, non-spherical shapes may be most efficient for providing the most amount of a chemical on a paramagnetic microparticle.

Qi, W. H., Wang, M. P., and Liu, Q. H. ((2005) Shape factor of nonspherical nanoparticles. J. Mater. Sci. 40, 2737-2739) refer to the relationship of the shape of nanoparticles to their surface area to volume ratios. They provide the term “shape factor” to compare the surface area to volume of non-spherical particles to spherical particles. They define the term shape factor as the ratio of the surface area of a non-spherical particle to that of a spherical particle, where both of the particles have identical volumes. Consequently, a particle with a shape factor of one (1) would have the same surface area to volume ratio as a spherical particle. Additionally, the non-spherical shapes may have an aspect ratio of between about 1:1 and 1:25.

We disclose tags that include paramagnetic microparticles that may have non-spherical shapes comprising an aspect ratio of between about 1:1 and 1:25. Additionally, we disclose paramagnetic particles with a shape factor of greater than one. In some examples, the paramagnetic microparticles may have the following shapes: tetrahedron, cube, octahedron, dodecahedron, capsule-shaped particle, icosahedron, ellipsoid, scalene ellipsoid, torus, toroidal polyhedron (genus two), toroidal polyhedron (genus three), cone, hexagonal prism, triangular prism, hexagonal pyramid, octagonal prism, cylinder, and stellated dodecahedron. In some embodiments, the paramagnetic microparticles may include villi-shaped extension which increase the surface area much like the villi of the small intestine. In some embodiments, the paramagnetic microparticles may have a porous surface. Chemicals may be connected to the paramagnetic microparticle within the pores as well as to the exterior surface to increase the amount of chemical per paramagnetic microparticle. In some embodiments, the paramagnetic microparticle may have an amorphous shape. In some embodiments, paramagnetic microparticles may be synthesized using microfluidic techniques to create the non-spherical shapes as described by Seo, K. D., Kim, D. S., and Sanchez ((2015) Lab Chip, 15, 3622-3226) which is hereby incorporated by reference in its entirety.

In some embodiments, the paramagnetic microparticles may be functionalized. The functional groups may be used to connect the unique and detectable chemicals to the paramagnetic microparticles. In some embodiments, the paramagnetic microparticles may include multiple different functional groups. Each different functional group may be used to connect a different unique and detectable chemical to the paramagnetic microparticles. Consequently, the paramagnetic microparticles include multiple attached chemicals, each chemical imparting a different piece of information about the tagged product.

In some embodiments, the at least one unique and detectable chemical includes a polynucleotide. The polynucleotide may include a sequence or series of sequences, each associated with a different category or piece of information about the pharmaceutical or nutritional product. In some embodiments, the polynucleotide is single stranded. In some embodiments the polynucleotide is DNA, RNA, or nucleotide analogs. In embodiments in which the polynucleotide is single stranded, the polynucleotide may be distinguished from other polynucleotides by hybridizing to a complementary single stranded polynucleotide.

In some embodiments, the unique and detectable chemical may include multiple different polynucleotides, each with different sequences which may be associated with different categories or information about the product. In some embodiments, the different polynucleotides are each attached to a different type of paramagnetic microparticle according to the disclosure while in other embodiments, polynucleotides with different sequences may be attached to the same type of paramagnetic microparticle.

In some embodiments, the paramagnetic microparticles may be coated with at least one ligand. Multiple ligands may be present on the same paramagnetic microparticle or different paramagnetic microparticles may each be coated with a different ligand and mixed. Each ligand may be associated with a different category or piece of information about the product. By adhering to their respective receptors or other binding proteins, the ligands may be identified or used to isolate the tags.

In some embodiments, the paramagnetic microparticles may be coated with at least one binding protein. Multiple binding proteins may be present on the same paramagnetic microparticle or different paramagnetic microparticles may each be coated with a different binding protein and mixed. By adhering to their respective ligands, each binding protein may be identified or used to isolate the tags.

The unique and detectable tags connected to the paramagnetic microparticles may be analyzed using a variety of analytical techniques known in the art. These may include light absorption spectroscopy. In some embodiments, the light absorption spectroscopy may include one or more of near infrared spectroscopy, visible spectroscopy, and ultraviolet spectroscopy.

The light absorption spectrometer or other analytical device may be part of a medical toilet. In this embodiment, the user may simply defecate into the bowl of the medical toilet as if the medical toilet were a traditional toilet. The medical toilet may isolate and/or analyze the tagged paramagnetic microparticles in the user's feces to gain information about pharmaceutical and nutritional products the user has ingested.

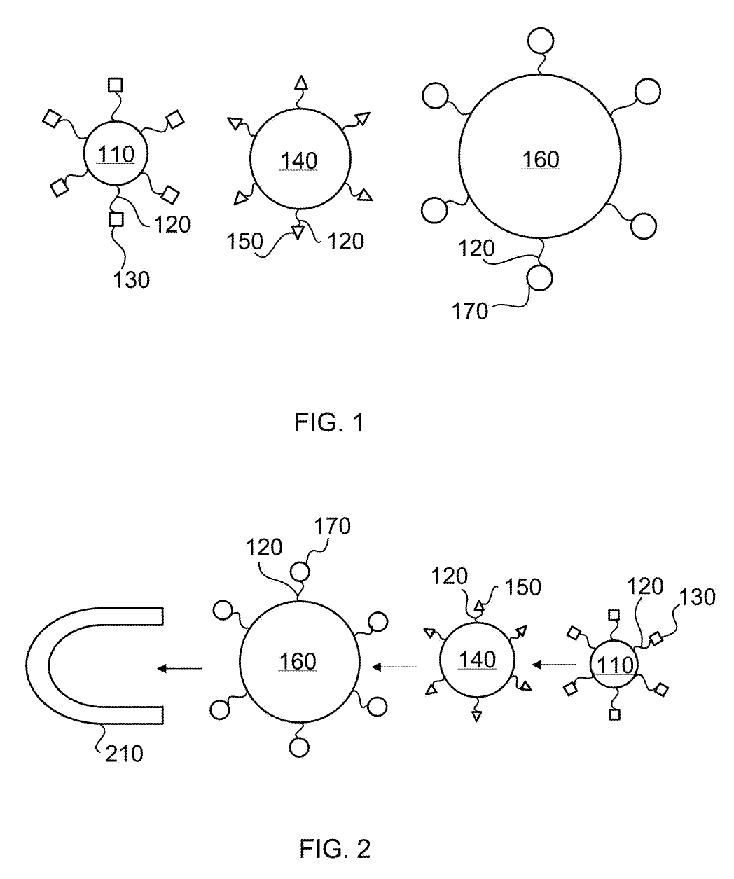

Referring now to the drawings, FIG. 1 illustrates cross sectional views of a series of paramagnetic microparticles which are shown in three different sizes and which possess three different masses and volumes. The cross-sections are taken at the widest points of a paramagnetic microparticle which may be a capsule shape. Microparticle 110 has the smallest diameter at its widest point and the smallest mass and volume. It is functionalized with functional group 120. Chemical marker 130 is connected to microparticle 110 through functional group 120. Microparticle 140 has an intermediate diameter at its widest point, an intermediate mass and volume, and is also functionalized with functional group 120. Chemical marker 150 is connected to microparticle 140 through functional group 120. Microparticle 160 has the largest diameter at its widest point and the largest mass and volume. It is also functionalized with functional group 120. Chemical marker 170 is connected to microparticle 160 through functional group 120. Each of chemical markers 130, 150, and 170 are associated with a different category or piece of information about a pharmaceutical or nutritional product. Upon separation of each of paramagnetic microparticles 110, 140, and 160 using an electromagnetic force, each of chemical markers 130, 150, and 170 may be detected, identified, and used to retrieve the associated information.

FIG. 2 illustrates the separation of the three paramagnetic microparticles of FIG. 1. Paramagnetic microparticles 110, 140, and 160 are shown in the presence of magnet 210. The electromagnetic force on each paramagnetic microparticle is proportional to its mass which is proportional to its mass and volume. Consequently, microparticle 160 (the largest of the three) moves toward magnet 210 faster than microparticle 140 and microparticle 140 moves toward magnet 210 faster than microparticle 110 (the smallest of the three).

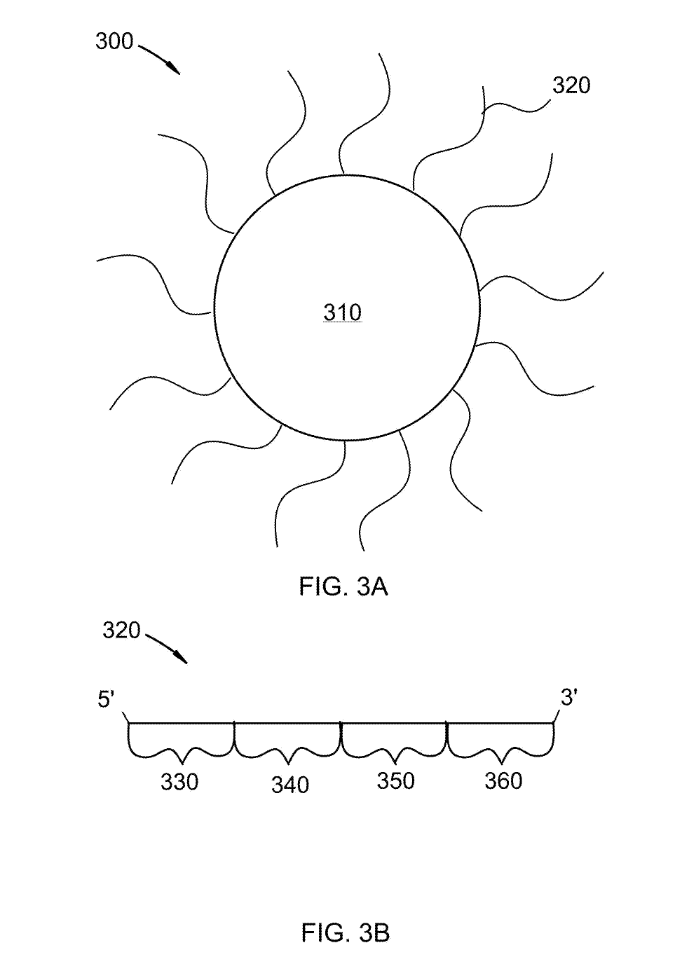

FIG. 3A illustrates a cross sectional view of tag 300. Tag 300 includes paramagnetic microparticle 310 which is connected to single-stranded polynucleotide 320. Additional single-stranded polynucleotides are illustrated but not labeled for purposes of clarity. Single-stranded polynucleotide 320 includes a sequence of nucleotides or nucleotide analogs which is associated with information about the pharmaceutical or nutritional product.

FIG. 3B is an expanded view of single-stranded polynucleotide 320. Single-stranded polynucleotide 320 is divided into sections 330, 340, 350, and 360. Each section includes a sequence of nucleotides or nucleotide analogs that is associated with a different category of information about a pharmaceutical or nutritional product.

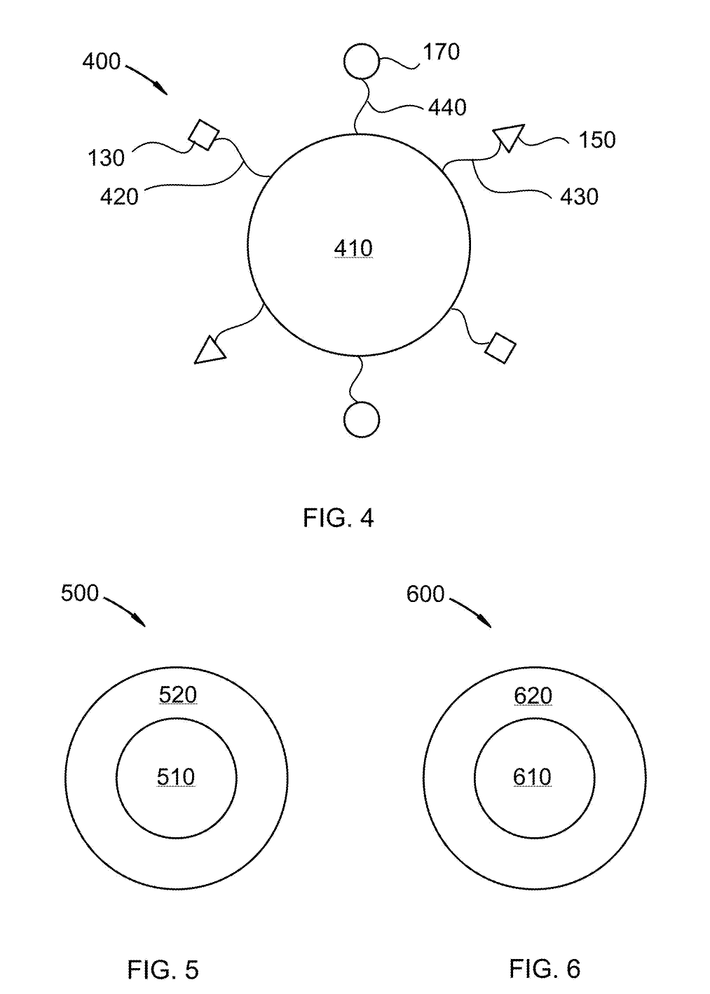

FIG. 4 illustrates a cross-sectional view of tag 400, which is connected to multiple chemical markers. Paramagnetic microparticle 410 is functionalized with three unique functional groups. Functional group 420 connects chemical marker 130 to paramagnetic microparticle 410, functional group 430 connects chemical marker 150 to paramagnetic microparticle 410, and functional group 440 connects chemical marker 170 to paramagnetic microparticle 410. Each of chemical markers 130, 150, and 170 are associated with a different category or piece of information about a pharmaceutical or nutritional product. Upon separation of a plurality of paramagnetic microparticles 410 using electromagnetic force, each of chemical markers 130, 150, and 170 may be detected, identified, and used to retrieve the associated information.

FIG. 5 illustrates a cross-sectional view of tag 500 which includes paramagnetic microparticle 510. Paramagnetic microparticle 510 is coated with ligand 520. Upon separation of tag 500 from a fecal sample, the identity of ligand 520 may be determined by exposing tag 500 to a series of binding proteins, one of which may be a binding protein for ligand 520.

FIG. 6 illustrates a cross-sectional view of tag 600 which includes paramagnetic microparticle 610. Paramagnetic microparticle 610 is coated with binding protein 620. Upon separation of tag 600 from a fecal sample, the identity of binding protein 620 may be determined by exposing tag 600 to a series of ligands, one of which may be a ligand for binding protein 620. This design is effectively the opposite of that described in FIG. 5 with the function and placement ligand and the binding protein reversed.

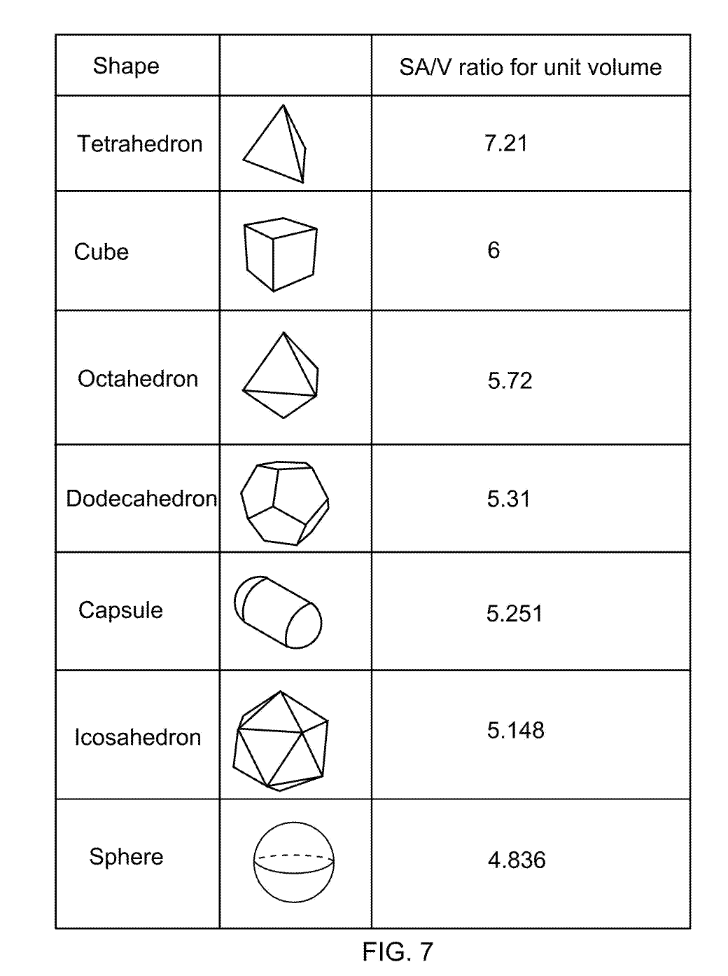

FIG. 7 shows a chart which illustrates several polyhedrons and their surface area to volume ratios. All have a greater surface area to volume ratio than a sphere which has a surface area to volume ratio of 4.836. The paramagnetic microparticles in the disclosed tags may include any of the non-spherical shapes of FIG. 7.

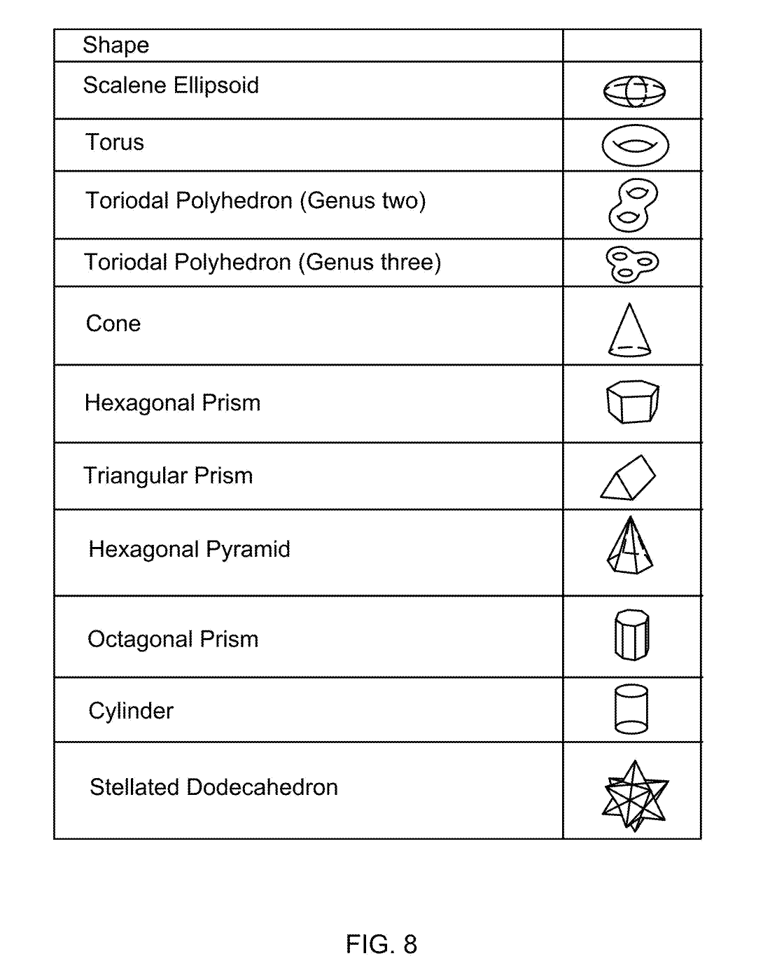

FIG. 8 illustrates additional shapes which the paramagnetic microparticles in the disclosed tags may include. All may have a greater surface area to volume ratio than a spherical shape.

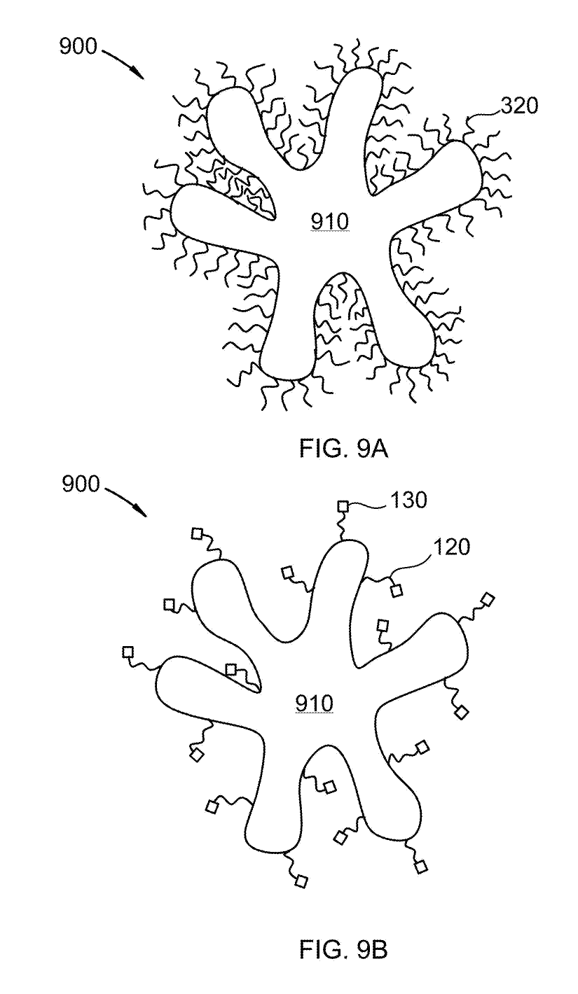

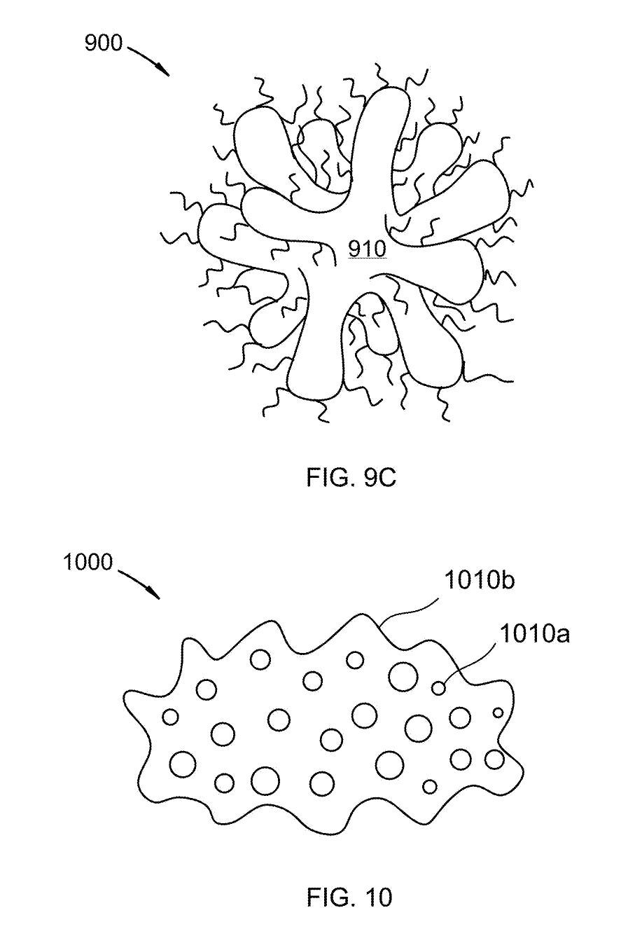

FIGS. 9A, 9B, and 9C illustrate embodiments of the disclosed tags which include paramagnetic microparticles having irregular shapes. FIG. 9A is a cross-sectional view of tag 900 which includes paramagnetic microparticle 910. Paramagnetic microparticle 910 includes villi-shaped extensions. Single-stranded DNA 310 is connected to the surface of one of the villi-shaped extensions of paramagnetic microparticle 910. Single-stranded DNA 310 may include a sequence that provides information about the product to which tag 900 is to be added as show in FIG. 3B.

The villi-shaped extensions increase the surface area of paramagnetic microparticle 910. The increased surface area increases the number of molecules of single-stranded DNA 310 which may be connected to the surface of paramagnetic microparticle 910 relative to that which would be possible with a smooth surface. Additional single-stranded DNA molecules are illustrated, but not numbered for purposes of clarity.

FIG. 9B is a cross-sectional view of another embodiment of tag 900 which has chemical 130 connected to the surface of paramagnetic microparticle 910 through functional group 120. Similar to the embodiment of FIG. 9A, the increased surface area provided by the villi-shaped extensions permits more molecules of chemical 130 to be connected to the surface of paramagnetic microparticle 910 than would be possible with a smooth surface. Additional molecules of chemical 130 and functional group 120 are illustrated, but not numbered for purposes of clarity. Chemical 130 provides information about the product to which tag 900 may be added.

FIG. 9C illustrates perspective view of tag 900 showing the villi-shaped extensions of paramagnetic microparticle 910. The villi-shaped extensions of paramagnetic microparticle 910 are shown extending in various directions from the center of paramagnetic microparticle 910. FIG. 9C shows the embodiment of FIG. 9A with single-stranded DNA molecules connected to the surface of paramagnetic microparticle 910.

FIG. 10 illustrates paramagnetic microparticle 1000 which may be used in the disclosed tag. Paramagnetic microparticle 1000 has an amorphous shape which may increase the surface area relative to a regular shaped particle. Paramagnetic microparticle 1000 also has a porous surface as shown by pores 1010a and 1010b. For purposes of clarity, other pores are illustrated but not numbered.

Chemicals may be connected to the exterior surface of paramagnetic microparticle 1000 and to the surface within pores 1010a and 1010b. The addition of surface within the pores increases the number of molecules of chemical which may be connected to paramagnetic microparticle 1000.

While specific embodiments have been described above, it is to be understood that the disclosure provided is not limited to the precise configuration, steps, and components disclosed. Various modifications, changes, and variations apparent to those of skill in the art may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

We claim:

1. A tag to identify pharmaceutical or nutritional products comprising at least one paramagnetic microparticle, wherein the at least one paramagnetic microparticle comprises:
a. a non-spherical shape;
b. a shape factor greater than one; and
c. at least one unique and detectable chemical, wherein the at least one unique and detectable chemical is connected to the at least one paramagnetic microparticle.

2. The tag of claim 1, wherein the non-spherical shape comprises a plurality of villi-shaped extensions.

3. The tag of claim 1, wherein the non-spherical shape consists of one or more of the following: an amorphous shape, a cube, an octahedron, a dodecahedron, a capsule, an icosahedron, an ellipsoid, a scalene ellipsoid, a torus, a toroidal polyhedron, a cone, a hexagonal prism, a triangular prism, a hexagonal pyramid, a cylinder, and a stellated dodecahedron.

4. The tag of claim 1, wherein the non-spherical shape comprises an irregular tetrahedron.

5. The tag of claim 1, wherein the at least one paramagnetic microparticle comprises a porous surface.

6. The tag of claim 1, wherein the at least one paramagnetic microparticle is functionalized, and wherein the at least one unique and detectable chemical is connected to the at least one paramagnetic microparticle through a functional group.

7. The tag of claim 6, wherein the at least one paramagnetic particle is functionalized with a plurality of unique functional groups, and wherein each of the plurality of unique functional groups is connected to a different unique and detectable chemical.

8. The tag of claim 1, wherein the at least one unique and detectable chemical comprises at least one polynucleotide, wherein each of the at least one polynucleotide comprises a sequence which is associated with information about the pharmaceutical or nutritional product.

9. The tag of claim 1, wherein the at least one paramagnetic microparticle is coated with a non-immunogenic substance.

10. The tag of claim 9, wherein the non-immunogenic substance comprises polyethylene glycol, poly(lactic-coglycolic acid), dendrimers, chitosan, sodium alginate, polyvinylpyrrolidone, copovidone, methylcellulose, cyclomethicones, or gold.

11. The tag of claim 1, wherein the at least one unique and detectable chemical is detectable with a light absorption spectroscopy technique.

12. The tag of claim 1, wherein the at least one unique and detectable chemical comprises at least one ligand, and wherein the at least one paramagnetic microparticle is coated with the at least one ligand.

13. The tag of claim 12, wherein the at least one ligand comprises a plurality of ligands.

14. The tag of claim 1, wherein the at least one unique and detectable chemical comprises a binding protein, and wherein the at least one paramagnetic microparticle is coated with the binding protein, wherein the binding protein binds to a ligand.

15. The tag of claim 1, wherein the at least one paramagnetic microparticle comprises a diameter of between about 5 μm and about 300 μm.

16. The tag of claim 1, wherein the at least one paramagnetic particle comprises a mass which is associated with a category, and wherein the category is associated with the identity of the pharmaceutical or nutritional product.

17. The tag of claim 16, wherein the category is selected from the following: a manufacturing site, a distributor, a dispensing pharmacy, a retailer, a prescribing healthcare provider, a formulation, a drug, a nutritional supplement, a food ingredient, and a prescribed end-user.

18. The tag of claim 1, wherein the at least one paramagnetic microparticle comprises a plurality of paramagnetic microparticles, and wherein the plurality of paramagnetic microparticles comprises a plurality of masses.

19. The tag of claim 18, wherein each of the plurality of paramagnetic microparticles comprising one of the plurality of masses is associated with a different unique and detectable chemical.

20. The tag of claim 1, wherein the non-spherical shape comprises an aspect ratio of between about 1:1 and 1:25.
